(19) United States
(12) Patent Application Publication
(10) Pub. No.: US 2004/0005291 A1
(43) Pub. Date: Jan. 8, 2004

(54) Methods for regulating co-stimulatory molecule expression with reactive oxygen

(76) Inventors: Martha Karen Newell Rogers, Colorado Springs, CO (US); Robert Camley, Colorado Springs, CO (US); Charles Richard Ill, Carlsbad, CA (US); Thomas Christensen, Colorado Springs, CO (US); Zbigniew Celinski, Colorado Springs, CO (US); Richard Trauger, San Diego, CA (US); Evan Newell, Toronto (CA); Elizabeth Villalobos-Menuey, Colorado Springs, CO (US)

Correspondence Address: Helen C. Lockhart, Wolf, Greenfield & Sacks, P.C., 600 Atlantic Avenue, Boston, MA, 02210, US

(21) Appl. No.: 10/272,432

(22) Filed: Oct. 15, 2002

Related U.S. Application Data
(60) Provisional application No. 60/329,477, filed on Oct. 14, 2001.
(60) Provisional application No. 60/329,280, filed on Oct. 12, 2001.

(51) Int. Cl. A61K 38/20; A61K 38/19; A61K 38/18; A61K 38/21; C12N 5/08; A61K 31/20

(52) U.S. Cl. 424/85.2; 424/85.6; 514/12; 514/560; 435/368; 424/85.1

(57) ABSTRACT

The invention is based in part on the discovery that the expression of co-stimulatory molecules such as B7.1, B7.2 or CD40 can be regulated using reactive oxygen species (ROS). Thus, the invention relates to methods of regulating co-stimulatory molecules by modulating reactive oxygen. The methods and products are useful, for example, for modulating antigen specific immune responses, treating disease, and for modulating cell growth.

### RELATED APPLICATIONS

This application claims priority to U.S. Provisional Patent Application filed Oct. 14, 2001, entitled “METHODS FOR REGULATING CO-STIMULATORY MOLECULE EXPRESSION WITH REACTIVE OXYGEN”, Serial No 60/329,477, and U.S. Provisional Patent Application filed Oct. 12, 2001, entitled “USE OF LOW FREQUENCY MICROWAVES AS A THERAPEUTIC”, Serial No. 60/329,280, each of which is incorporated by reference in its entirety.

### FIELD OF THE INVENTION

This invention relates to methods of regulating co-stimulatory molecules by modulating reactive oxygen and related products. The methods and products are useful, for example, for tissue generation, transplantation, modulating antigen specific immune responses, treating disease such as cancer, and for modulating cell growth.

### BACKGROUND OF THE INVENTION

One important method of regulating disease through the manipulation of cell growth and proliferation involves immune cells. The immune system, a complex organization of cells, tissues and organs, serves to protect us from potential harm. Extraordinary advances in our understanding of the immune system have been made in the last hundred years, especially in the time since the discovery of the T cell and B cell. Nonetheless, fundamental questions remain unanswered. One of these concerns the nature of immune-privilege. It is widely accepted that certain tissues (brain, eye, ovary, testes) interact differently with the immune system compared to most other tissues. These tissues are commonly termed immune-privileged sites, however the basis for the privilege is unknown.

The complex process of T-cell activation and proliferation is based on diverse interactions such as antigen presentation, cell-cell contact and soluble immune mediators e.g., cytokines or lymphokines. Many of these interactions are mediated in T-cells through surface receptors. T helper cells, for example, require for activation both the presentation of an antigen by an antigen presenting cell (APC) in association with major histocompatibility complex (MHC) and a secondary signal. The secondary signal may be a soluble factor or may involve an interaction with another set of receptors on the surface of T-cells. Antigen presentation in the absence of the secondary signal, however, is not sufficient to activate T helper cells.

The first, recognition of antigen and Major Histocompatibility Complex-encoded (MHC) molecules has been studied extensively (Marrack P, Kappler J. The T cell receptor. Science 1987; 238:1073-1079). In contrast, the controlling mechanism for the second signal (co-stimulation), predicted by Bretscher and Cohn in 1971 (Bretscher Pa., Cohn M. A theory of self discrimination. Science 1970;169: 1042-1049) and confirmed by discovery of B7/CD28 family members (Linsley P, and Ledbetter J A. Role of the CD28 receptor during T cell responses to antigen. Annual Review in Immunology 1993;11:191-212; Linsley P S, et al. Human B7-1 (CD80) and B7-2 (CD86) bind with similar activities but distinct kinetics to CD28 and CTLA4. Immunity 1994;1:793-801; June C H, et al. The B7 and CD28 receptor families. Immunol. Today 1994;15:321-330; Kuchroo V K, et al. B7-1 and B7-2 costimulatory molecules activate differentially the Th1/Th2 developmental pathways: application to autoimmune disease therapy. Cell 1995;80:707-718; and Lanier L L, et al. CD80(B7) and CD86(B70) provide similar costimulatory signals for T cell proliferation, cytokine production, and generation of CTL. Journal of Immunology 1995;154:97-105) in the 1990s, is not known. T-cells travel through the body looking for antigens, MHC, and a costimulatory signal. In the absence of activation T-cells ignore the tissue. If the T cell is activated the consequences can be: 1) the destruction of the damaged cells or 2) the repair of damaged cells by promoting regeneration either directly or indirectly.

### SUMMARY OF THE INVENTION

The invention involves the finding that the presence or absence of reactive oxygen species (ROS) plays a role in regulating the expression of co-stimulatory molecules such as B7.1, B7.2 and/or CD40. The method involves in some aspects a method for inhibiting co-stimulatory molecule expression in a cell by decreasing exposure of a cell to a ROS to inhibit co-stimulatory molecule expression in the cell. In some embodiments the cell is a stem cell or the cell may be, for instance, a cell derived from skin, heart, liver, or kidney. In other embodiments the co-stimulatory molecule is B7.1, B7.2 and/or CD40.

In some embodiments the cell is implanted into a subject. In other embodiments the cell is grown in vitro under growth conditions prior to implantation. The method may be performed in vitro or in vivo to a subject such that the ROS is decreased by contacting the cell with an inhibitor of ROS or by administering to the subject an inhibitor of ROS or by administering the treated cell to the subject. In other embodiments the inhibitor of the ROS is a compound selected from the group consisting of glutathione S reductase, glutathione, Copper/Zinc superoxide dismutase, and Manganese superoxide dismutase.

According to other aspects, the invention is a method for inducing co-stimulatory molecule expression in a cell by increasing exposure of a cell to a ROS to induce co-stimulatory molecule expression in the cell. In some embodiments the cell is a T cell, a nerve cell or a neutrophil. In another embodiment the co-stimulatory molecule is B7.1, B7.2 and/or CD40.

In some embodiments the cell is exposed to growth conditions to promote cell proliferation. Growth conditions include but are not limited to exposure to at least one of insulin, nerve growth factor, fibroblast growth factor, platelet derived growth factor, erythropoietin, and cytokines such as IL-2, IL-4, γ interferon, α and β interferons, TNF (tumor necrosis factor) α, TGF (T-cell growth factor) α and β, and lymphotoxin.

The method may be performed in vitro or in vivo to a subject such that the ROS is increased by contacting the cell with a ROS or by administering to the subject a ROS or by administering the treated cell to the subject. In some embodiments the cell is contacted with an activator of ROS or the subject is administered an activator of ROS. Optionally the activator of ROS is an inhibitor of mitochondrial electron transport, an inhibitor of glutathione or glutathione S reductase, an inhibitor of superoxide dismutase, or an inhibitor of lysosomal UCP. The inhibitor of mitochondrial electron transport may be a compound selected from the group consisting of reactive oxygen species, angiostatins, angiogenics, viral components, and exposure to sub-toxic microwaves or low dose radiation. Optionally the viral component is a gene product such as HIV Nef, HIV tat, or adenoviral E1B. In other embodiments the activator of ROS is exposure to microwaves.

In other embodiments the method also involves contacting the cell or the subject with an antigen. Optionally the antigen is selected from the group consisting of a tumor, a viral, a bacterial, a parasitic, and a fungal antigen.

In yet other aspects the invention is a method for modulating B7.1, B7.2 and/or CD40 expression on embryonic stem cells by contacting an embryonic stem cell with a compound for modulating ROS to modulate B7.1, B7.2 and/or CD40 expression on the embryonic stem cell. In an embodiment the compound for modulating ROS is an inhibitor of ROS. In another embodiment the compound for modulation ROS is a reactive oxygen species. Optionally the embryonic stem cell may be administered to a subject.

A method for promoting nerve cell generation is provided according to other aspects of the invention. The method involves contacting a nerve cell with a neural cell ROS activator in an effective amount to promote differentiation and/or growth. In some embodiments the neural cell ROS activator is a reactive oxygen species, angiostatins, angiogenics, viral components, or exposure to sub-toxic microwaves or low dose radiation. In other embodiments the nerve cell may be contacted with a neural activating cell.

In other aspects the invention is a method for promoting non-neural tissue generation by contacting a non-neural tissue with an activator of ROS in an effective amount to induce co-stimulatory molecule expression on the surface of cells of the tissue, and exposing the tissue to growth conditions to promote generation of the tissue. In some embodiments the ROS activator is y interferon, lipoproteins, fatty acids, cAMP inducing agents, a UCP expression vector, a B7.1, B7.2 and/or CD40 expression vector, angiostatins, angiogenics, viral components, or exposure to sub-toxic microwaves or low dose radiation. The growth conditions may include exposure to at least one of insulin, fibroblast growth factor, platelet derived growth factor, erythropoietin, and cytokines such as IL-2, IL-4, γ interferon, α and β interferons, TNF (tumor necrosis factor) α, TGF (T-cell growth factor) α and β, and lymphotoxin. In some embodiments the non-neural tissue is selected from the group consisting of kidney, lung, pancreas, skin.

In the methods described herein a further step may involve exposing the non-neural tissue or the nerve cell to a T cell. The non-neural tissue or the nerve cell is exposed to the T cell in vitro or in vivo. If the exposure is in vitro the non-neural tissue or the nerve cell may be implanted in a subject after exposure to the T cell. Optionally the T cell is a cell of the subject. The non-neural tissue or the nerve cell may be autologous tissue or the non-neural tissue or the nerve tissue may be a donor organ.

The biopsy of the non-neural tissue or the nerve tissue may be removed from a subject and exposed to a T cell of the subject. Then, the T cell may be returned to the subject after exposure to the biopsy.

The methods may also involve a step of contacting the nerve or non-neural tissue with a receptor for a co-stimulatory molecule.

In other aspects the invention relates to a method for transplanting an organ into a recipient subject by treating a donor organ with an inhibitor of ROS in an effective amount to reduce costimulatory molecule expression on cells of the donor organ, and transplanting the donor organ into the recipient subject.

A method for treating autoimmune disease by administering to a subject having or at risk of developing an autoimmune disease an inhibitor of ROS in an effective amount to reduce costimulatory molecule expression on target autoimmune cells in order to treat the autoimmune disease is also provided. In some embodiments the autoimmune disease is multiple sclerosis.

The inhibitor of ROS may be a compound which activates or induces glutathione S reductase, glutathione, Copper/Zinc superoxide dismutase, or Manganese superoxide dismutase.

The invention in other aspects is a method for treating cancer by exposing cancer cells of a subject to sub-toxic levels of microwave or to sub-toxic levels H₂O₂ in an effective amount to induce expression of a co-stimulatory molecule on the surface of the cancer cells and contacting the cell with an agent to kill the cell in order to treat the cancer. In some embodiments the cancer cells are exposed to 2-deoxyglucose or analogs thereof. In other embodiments the agent is a co-stimulatory molecule receptor, such as co-stimulatory molecule receptor on an immune cell or a soluble receptor. The agent in other embodiments is a sub-cytotoxic dose of an anti-cancer drug, such as radiation.

Each of the limitations of the invention can encompass various embodiments of the invention. It is, therefore, anticipated that each of the limitations of the invention involving any one element or combinations of elements can be included in each method and product.

### BRIEF DESCRIPTION OF THE FIGURES

The present invention may be more easily and completely understood when taken in conjunction with the accompanying figures.

FIG. 1 is a bar graph depicting data demonstrating that low frequency, low intensity microwaves induce increases in intracellular reactive oxygen and increases in cell surface expression of B7.2 on MCF7, human breast cancer cell lines.

FIG. 2 is a bar graph depicting data demonstrating that exogenous H₂0₂ directly induces increased cell surface expression of B7.2 on pro-myelocyte lines U937 and HL60 cells and in neural PC12 and PC12Trk neural cell lines.

FIG. 3 is a set of graphs depicting data demonstrating that exogenous H₂0₂ directly induces increased cell surface expression of fas (3A) and B7.2 (3B) on keratinocytes.

FIG. 4 is a graph depicting the effects of ethanol in neural stem cells. The cells were stained with DCF-DA.

### DETAILED DESCRIPTION

The invention is methods and products relating to the control of co-stimulatory molecule expression on the surface of a cell. It was discovered according to some aspects of the invention that the expression of co-stimulatory molecules, and in particular B7, is integrally related to the presence or absence of reactive oxygen species (ROS). In general it was found that in the presence of increasing amounts of ROS co-stimulatory molecule expression is induced and that if ROS is decreased the expression of co-stimulatory molecules is also decreased. The ability to regulate ROS levels in order to change the expression of co-stimulatory molecules has important implications for many diseases as well as therapeutic and prophylactic therapies.

The energy metabolism of a cell is also a key factor for determining how the immune system interacts with that cell. In cells there are a limited number of metabolic states, depending on the fuel the cell consumes. These include glucose (carbohydrates), lipids (fats), and proteins. In particular, it has been discovered that the ability to efficiently use fat for fuel in combination with the expression of particular cell surface molecules confers immune privilege. Uncoupling proteins play an important role in this mechanism because they are instrumental in the fat burning process. As a result, changes in metabolism (caused by stresses, fuel availability, age, hormones, radiation, drugs, etc.) necessarily produce changes in the immune response. This has profound implications for controlling autoimmune diseases, preventing graft rejection, promoting tissue generation, and targeting tumor cells for destruction.

The implications of this connection between cell metabolism and how a cell is recognized by the T cells are profound because T cell recognition and activation are fundamental to the operation of the immune system. The findings described herein form the basis for methods involving the manipulation of how T cells recognize a cell e.g., by changing the metabolism of the cell being recognized, in order to direct the immune system to ignore, destroy, repair, or regenerate the recognized cell. The invention described herein demonstrates an intimate connection between cellular energetics and how the immune system responds to an individual cell.

We have recognized that the choice of fuel (e.g., glucose and/or lipid) for mitochondrial metabolism is part of a metabolic behavior that regulates the interaction of the cell with any other cell including cells of the immune system. Our findings indicate that there are at least three metabolic base states and that these base states are defined by the levels of reactive oxygen inside the cell. The levels of reactive oxygen impact whether a tissue is ignored by the immune system (referred to herein as a growth inhibited state), the tissue undergoes regenerative growth nurtured by the immune system (referred to herein as a growth induced state), or the tissue is sensitive to immune induced death i.e. as would happen to infected or severely damaged cells (referred to herein as an immune targeted state).

Cells in the immune targeted state have high intracellular levels of reactive oxygen. Under these conditions co-stimulatory molecules are expressed under conditions which lead to rejection of the tissue. For instance the following conditions produce cells in the immune targeted state that can be targeted for destruction: high levels of intracellular reactive oxygen induced under conditions in which no additional metabolic strategy can deal with it. For example, uncoupling proteins cannot be effectively expressed, the expression of uncoupling proteins has been disabled by drugs which interrupt UCP expression or activity such as anti-sense to UCP, or the uncoupled, protective metabolic state has been negatively affected by metabolic interference from such compounds as chemotherapeutic agents (i.e., adriamycin, 5FU, methotrexate, trimetrexate, cisplatin, etc. at concentrations greater than 10-8 M in vivo), radiation of any kind at levels greater that 25 to 30 grey, high intensity, high frequency microwaves, gamma radiation above 25 grey. Additionally, conditions which disable other protective strategies, such as manganese or copper/zinc superoxide dismutase, glutathione-S reductase, etc. (i.e. inhibitors of such compounds) could tip the balance to a metabolic strategy in which the levels of reactive oxygen are high enough to trigger destructive immune recognition, particularly in the absence of growth signals which tip the balance towards the growth induced state. Another example of conditions causing high reactive oxygen leading to the immune targeted state involves a combined approach of two strategies resulting in high intracellular reactive oxygen such as, for example, lower level radiation (10 to 25 grey) with less that 10-8 M chemotherapeutic.

Cells in the growth induced state have intermediate levels of intracellular reactive oxygen causing an induction in co-stimulatory molecules. These cells are maintained, preferably during exposure to the immune system, under growth conditions, such that the cells are encouraged to grow. Growth promoting conditions include but are not limited to the following: insulin (e.g., for modulation growth of brain, eye, skin, muscle, kidney, etc); nerve growth factor; fibroblast growth factor (e.g., for modulating growth of connective tissues); platelet derived growth factor (e.g., for modulating growth of platelets); erythropoietin (e.g., for modulating red blood generation); and cytokines including such as IL-2, IL-4, γ interferon, α and β interferons, TNF (tumor necrosis factor) α, TGF (T-cell growth factor) α and β, and lymphotoxin.

Thus methods for producing cells in the growth induced state involve generating an intermediate level of reactive oxygen under growth conditions. Agents that are useful for tipping the balance of the metabolic state of the cell to increase reactive oxygen to a sufficient level are those compounds referred to herein (and described in more detail below) as activators of ROS. Generally these compounds act to promote increased gene expression of UCP, MnSOD, glutathione S reductase to produce a level of intracellular reactive oxygen which when combined with the growth promoting environment can promote regenerative repair. These moderate levels of intracellular reactive oxygen produced by these conditions prime the cells for repair. An example of agents that produce moderate or intermediate levels of reactive oxygen include but are not limited to sub-cytotoxic doses of H202 (25 mM or less), low levels of gamma radiation (1 to 20 grey), low intensity, low frequency microwaves (e.g. 10 mV).

Cells in the growth inhibited state are immune-privileged cells. These cells are maintained under conditions in which lipids are preferentially used for fuel. The cells have lower mitochondrial membrane potential, are less likely to have surface MHC, are less easily damaged by free radicals, and have relatively lower levels of (or no) costimulatory molecule expression. Cells in this state are not recognized by the immune system. The levels of reactive oxygen in the cell should be maintained carefully. At lows levels of reactive oxygen the cells develop a “spore-like” state that permits them to avoid recognition and rejection by the immune cells. Cells in this state may be valuable for storing stem cells or preserving grafts prior to implantation. In addition to maintaining low levels of reactive oxygen, e.g. by using inhibitors of ROS, the state is optimally achieved under conditions of low glucose, low electromagnetic radiation, and the presence of non-glucose carbon sources, such as polyunsaturated fatty acids.

A major problem in transplantation of organs is immunological rejection. It is now possible to alter the cells being transplanted in a way that removes or reduces the co-stimulatory signal (i.e., producing a growth inhibited state). This can be accomplished according to the invention without (or in combination with) the use of general immunosuppressive agents. Another important class of diseases in which it is desirable to cause the T cells to ignore a tissue is autoimmune diseases such as multiple sclerosis (MS), systemic lupus erythematosus (SLE), and rheumatoid arthritis. In these diseases it is important to direct the immune system to avoid attacking self tissue. In autoimmune conditions the MHC signal is present. This is the reason why the immune system is prompted to attack the tissue. Using the methods of the invention it is possible to reduce or eliminate the co-stimulatory signal (the “danger signal”) in order to reduce the damage caused by the immune system. In contrast, making changes in cellular metabolic activity to produce an immune targeted state can promote recognition and destruction of tumor cells. Additionally induced repair and regeneration of tissues is important in many contexts and can be achieved by causing the cells to assume a growth induced state. For instance, regeneration of neurons is most important in helping stroke victims or people with spinal cord injuries.

The invention involves in vitro, in vivo, and ex vivo technologies. The in vitro methods of the invention are useful for a variety of purposes. For instance, the methods of the invention may be useful for manipulating the expression of co-stimulatory molecules on cells cultured in vitro for studies involving immune recognition as well as for manipulating the culture conditions.

In addition to the in vitro methods, the methods of the invention may be performed in vivo or ex vivo in a subject to manipulate one or more cell types within the subject. An “ex vivo” method as used herein is a method which involves isolation of a cell from a subject, manipulation of the cell outside of the body, and reimplantation of the manipulated cell into the subject. The ex vivo procedure may be used on autologous or heterologous cells, but is preferably used on autologous cells. In some embodiments, the ex vivo method is performed on cells that are isolated from bodily fluids such as peripheral blood or bone marrow, but may be isolated from any source of cells. When returned to the subject, the manipulated cell will have increased or decreased expression of co-stimulatory molecules (or mechanisms for inducible variations in expression), depending on the treatment to which it was exposed. Ex vivo manipulation of cells has been described in several references in the art, including Engleman, E. G., 1997, Cytotechnology, 25:1; Van Schooten, W., et al., 1997, Molecular Medicine Today, June, 255; Steinman, R. M., 1996, Experimental Hematology, 24, 849; and Gluckman, J. C., 1997, Cytokines, Cellular and Molecular Therapy, 3:187. The ex vivo activation of cells of the invention may be performed by routine ex vivo manipulation steps known in the art. In vivo methods are also well known in the art. The invention thus is useful for therapeutic purposes and also is useful for research purposes such as testing in animal or in vitro models of medical, physiological or metabolic pathways or conditions.

The methods of the invention are useful in subjects. A subject as used herein means vertebrates such as humans, primates, horses, cows, pigs, sheep, goats, dogs, cats and rodents.

The complex process of immune cell activation and proliferation is based on diverse interactions such as antigen presentation, cell-cell contact and soluble immune mediators e.g., cytokines or lymphokines. Many of these interactions are mediated in T- and other immune cells through surface receptors. T helper cells, for example, require for activation both the presentation of an antigen by an antigen presenting cell (APC) in association with major histocompatibility complex (MHC) and a secondary signal. The secondary signal may be a soluble factor or may involve an interaction with another set of receptors on the surface of T- and other immune cells. Antigen presentation in the absence of the secondary signal, however, is not sufficient to activate T helper cells. The secondary signals described herein are referred to as co-stimulatory molecules.

A co-stimulatory molecule as used herein refers to a molecule such as B7, CD40 etc expressed on the surface of a cell and which is capable of interacting with a receptor on the surface of an immune cell. Receptors for co-stimulatory molecules include but are not limited to fas ligand, CD28, CTLA4, and CD40 ligand. When used according to methods of the inventions, the receptors for co-stimulatory molecules may be soluble receptors or fragments thereof which interact with the co-stimulatory molecule and induce the secondary signal process or may be cell surface receptors. The receptors are generally found on the surface of cells such as CD4 T cells, CD8 T cells, NK cells, gamma delta T cells, dendritic cells, B cells and macrophage. In addition to these cells, cells expressing such receptors or functional fragments thereof may be generated using routine procedures known in the art such as transfection.

The CTLA-4/CD28/B7 system is a group of proteins involved in regulating T-cell proliferation through this secondary signaling pathway. The T-cell proliferative response is controlled by the interaction of the B7 family of proteins, which are expressed on the surface of APCs, with CTLA-4 (cytotoxic T lymphocyte antigen #4) and CD28.

The B7 family of proteins is composed of structurally related glycoproteins including B7-1, B7-2, and B7-3 (Galea-Lauri et al., Cancer Gene Therapy, v. 3, p. 202-213 (1996); Boussiotis, et al., Proc. Nat. Acad. Sci. USA, v. 90, p.11059-11063 (1993)). The different B7 proteins appear to have different expression patterns on the surface of antigen presenting cells. For example B7-2 is constitutively expressed on the surface of monocytes, whereas B7-1 is not, although B7-1 expression is induced in these cells when the cells are stimulated with interferon gamma (IFN-γ). The different expression patterns may indicate a different role for each of the B7 family members. The B7 proteins are believed to be involved in the events relating to stimulation of an immune response by its ability to interact with various immune cell surface receptors. It is believed, for example, that B7 plays a role in augmenting T-cell proliferation and cytokine production through its interaction with the CD28 receptor.

CD28, a homodimeric glycoprotein having two disulfide linked 44-kd subunits, is found on 95% of CD4⁺ and 50% of CD8⁺ cells. Studies using monoclonal antibodies reactive with CD28 have demonstrated that CD28 is involved in a secondary signal pathway in the activation of T-cell proliferation. Antibodies which block the interaction of CD28 with its ligand have been found to inhibit T-cell proliferation in vitro resulting in antigen specific T cell anergy. (Harding et al., Nature, v. 356, p. 607 (1991)).

A T-cell surface receptor protein having approximately 20% sequence homology to CD28 is CTLA-4. Although CTLA-4 is not endogenously expressed on T-cell surfaces, its expression is induced when CD28 interacts with B7 on the surface of an APC. Once CTLA-4 is expressed on the surface of the T-cell it is capable of interacting with B7.

Thus, the invention relates in some aspects to methods for promoting tissue generation. Tissue generation as used herein refers to the induction of differentiation and or growth. For instance, stem cells may be treated to induce nerve cell generation. Such cells can differentiate into neural cells under the appropriate conditions. Additionally, tissue generation refers to the proliferation of cells, such as organ tissue, when it is desirable to generate new or repair existing organs.

A method for promoting nerve cell generation is provided. Nerve cells can be induced to express co-stimulatory molecules and can interact with T- and other immune cells through receptors. The co-stimulatory molecule on the nerve cell surface can engage the receptor on the immune cell surface to co-stimulate the immune cell, leading to activation of the immune cell. The activated immune cell may then release nerve growth factor which stimulates the nerve cell.

According to a method of the invention nerve cell is exposed to a neural activating cell. A “neural activating cell” as used herein, is a cell which is capable of producing nerve growth factor when activated and which includes a cell surface B7 (or other co-stimulatory molecule) receptor. For instance, B7 receptors include CD28 and CTLA-4. Many cells which are the neural activating cells of the invention have been described in the art. These cells include, for example, T cells (including both gamma, delta and alpha-beta T cells), macrophage, dendritic cells, CTLA-4 or CD-28 expressing B cells.

A “B7 receptor” as used herein is a cell surface immune molecule which interacts with B7 on a partner cell and causes activation of the cell on which it is expressed. Preferably the B7 receptor is a CD28 molecule or a CTLA4 molecule.

The nerve cell is exposed to the neural activating cell to cause differentiation of the nerve cell. The step of exposing can be performed in vitro, by simply mixing the two populations of cells, the nerve cell and the neural activating cell. It can be accomplished in vivo by causing the accumulation of the neural activating cells in the local environment of the nerve cell. For instance, the neural activating cells may be implanted, or the local environment may be manipulated to cause accumulation of the neural activating cell. For instance, stimulating an immune response in the local environment would cause the accumulation of T cells, B cells, dendritic cells and macrophage. The neural activating cell may also be a cell which produces nerve growth factor upon activation and which is engineered to express a B7 receptor on its surface, e.g. by transfection with an inducible or constitutively expressed B7 receptor gene, such as by the methods described above.

The methods of the invention in some aspects may also be performed using an endogenous neural activating cell. For instance the endogenous neural activating cell may be a cell having a cell surface B7 receptor, such as CD28 and CTLA-4. In this case the method would only include the step of contacting a nerve cell with an amount of a B7 inducing agent effective to induce the expression of B7 on the surface of the nerve cell in the presence of a neural activating cell.

When the neural activating cell is a cell having a cell surface B7 receptor which is already present in interactive proximity to the B7, the cell does not have to be manually brought into contact with the B7 on the nerve cell.

When the nerve cell is exposed to a neural activating cell the cell surface B7 can interact with the B7 receptor to activate the neural activating cell. Once activated, the neural activating cell produces and releases nerve growth factor into the local environment. This locally produced nerve growth factor is capable of causing the nerve cell to become differentiated. Although the invention is not limited to a specific mechanism of action, applicants believe that the mechanism through which neuro-differentiation occurs is that the nerve growth factor interacts with the nerve cell surface nerve growth factor receptor such as Trk. It is also believed that engagement of the B7 on the cell surface or the induction thereof causes the expression of nerve growth factor receptors on the surface of the nerve.

In one embodiment of the invention, the receptors for nerve growth factor may be induced to be expressed on the surface of the nerve cell. Two known nerve growth factors are tyrosine, kinase A (TrkA) and p75NGRF. When these receptors interact with nerve growth factor on the surface of a nerve cell, it stimulates the cell to undergo neuronal differentiation. Expression of these receptors on the surface of the nerve cell may be performed by any method known in the art. For instance, the nerve cell may be recombinantly engineered to constitutively or inducibly express the DNA for these receptors, such as by the methods described above.

Nerve growth factor (NGF), originally described by Levi-Montalcini and Hamburger in 1953, contains two copies of three types of polypeptides and exhibits approximately 50% of homology with other neurotrophins i.e., brain-derived neurotrophic factor (BDNF), NT-3, NT-4 and NT-5. It binds to tyrosine kinase A (TrkA) and p75NGF receptors in a synergistic manner. Tyrosine kinase B (TrkB) and tyrosine kinase C (TrkC) receptors preferentially bind BDNf and NT-3 respectively. Intracellular signal proteins via Src homology 2 (SH20 domain interactions such as phospholipase C and the p85 sub-unit of phosphatidyl-inositol 3-kinase bind to the tyrosine-phosphorylated receptors and allow multimeric protein complexes to form and lead to the activation of specific signal transduction pathways.

Nerve cells express molecules which are requisite for T cell activation, indicating that there is a neuro-immunological intercellular interactive component that occurs during neuronal differentiation. NGF and EGF have profound effects on the differentiation process in utero and early life and on the regeneration process after pathologic damage.

Another aspect of the invention is a method for reinnervating an injured tissue. The method involves the step of contacting a nerve cell in the injured tissue with an activator of ROS, wherein the treated nerve cell will undergo neuronal differentiation in the presence of a neural activating cell in the injured tissue to reinnervate the injured tissue. The nerve cell may be treated in vivo or may be manipulated in vitro and then transplanted. Methods are known in the art for implanting nerve cells into living tissue. For example, nerves can be implanted directly into exposed tissue or may be implanted in biodegradable tubes which will guide the extension of the nerve into surrounding tissue where it can be differentiated.

An injured tissue is a tissue in which nerve damage has been sustained. An injured tissue may include for example, a spinal chord injury, a severed or severely damaged limb or any other tissue which can be innervated and in which the nerve has been damaged. Neural activating cells are generally found in skin and muscle surrounding the nerves of an injured tissue. These neural activating cells can stimulate the differentiation of the nerve cell once they are activated by interaction with the B7 on the surface of the nerve cell.

The invention also includes a method for treating a neurodegenerative disorder by administering an amount of an activator of ROS effective to induce the expression of B7 on the surface of a nerve cell. A “neurodegenerative disorder” as used herein, is a disorder associated with the death or injury of neuronal cells. For example, the loss of dopaminergic neurons in the substantia nigra ultimately leads to Parkinson's Disease. The deposition of β-amyloid protein in the brain generally causes neural damage leading to Alzheimer's Disease. Conditions involving injuries such as brain ischemia, spinal chord damage, and severance of limbs often causes extensive neuronal cell death. When a nerve is severed, the regions of the nerve cells which are distal to the severance become separated from the nerve cell body and degenerate. After such a severance, it is possible for the nerve cell body to regenerate by re-extension of the severed axons. This process of nerve regeneration does not occur naturally in the absence of certain environmental conditions. In some cases in the prior art, various factors such as nerve growth factor have been added to the nerve to attempt to stimulate the regeneration. The methods of the invention describe a different system in which the nerve cell is manipulated to express an immune recognition molecule on its surface which can then cause the local expression of nerve growth factor leading to differentiation. This method more closely simulates the natural processes of neuronal regeneration. Other neurodegenerative diseases include for example but are not limited to epileptic seizures and amyotrophic lateral sclerosis.

Another aspect of the invention involves a method for inducing apoptosis in a nerve cell. The method involves the step of contacting a nerve cell with an amount of an inhibitor of ROS which when exposed to a nerve cell causes down regulation of B7 expression and contacting a neural activating cell with an amount of a B7 receptor blocking agent effective for inducing apoptosis in the nerve cell.

A “B7 receptor blocking agent” as used herein is any agent which interacts with a B7 receptor but does not cause activation of the cell and prevents that receptor from binding to B7. These agents include, for example, but are not limited to anti-CD28 antibodies, CD28 binding peptides, anti-CTLA-4 antibodies, CTLA-4 analogs and CTLA-4 binding peptides which do not cause activation of the receptor. Other B7 receptor blocking agents can be identified by those of skill in the art by routine experimentation using immune cell activation assays such as a T cell activation assay.

This method is useful whenever it is desirable to induce apoptosis of a nerve cell. For instance, it may be useful to induce apoptosis of a nerve cell in vitro in order to screen molecules for their ability to prevent apoptosis of nerve cells. Other uses will be apparent to those of ordinary skill in the art.

The invention also relates to methods for facilitating repair or generating other types of tissue for transplantation or in vivo methods, such as wound healing or tissue growth. The methods may be performed on any type of tissue using the conditions described herein related to manipulation of cells in the growth induced state. For instance cells may be treated with an ROS activator under growth promoting conditions to cause the cells to express cell surface co-stimulatory molecules but to maintain a metabolic state that causes the immune system to recognize the cell as a cell undergoing growth rather than marked for destruction.

The methods are useful for generating tissues in vitro or in vivo. For instance a tissue or a piece of a tissue may be isolated from a subject and maintained in vitro under growth conditions and contacted with a ROS activator. Alternatively a tissue in need of regeneration may be treated in vivo with a ROS activator under growth promoting conditions.

Preferably the cells grown under these conditions may be exposed to immune cells to promote recognition of the immune cells of the particular metabolic state. In one example an organ or biopsy of an organ may be cultured in vitro under the appropriate conditions. A sample of T cells may be isolated from a recipient subject. The T cells may be mixed with the organ or some cells of the organ. The T cells may then be administered to the subject in conjunction with the transplantation of the organ if the organ was grown in vitro or alone if the organ was treated in vivo.

The findings of the invention are also useful for the manipulation of stem cells or other hematopoietic cells. It has been discovered that the levels of co-stimulatory molecules on stem cells can be manipulated by altering the levels of ROS. In the presence of increased levels of ROS the expression of co-stimulatory molecules such as B7 are induced on stem cells. Thus, the invention in some aspects encompasses mechanisms for controlling these complex interactions to regulate local levels of ROS and thus the processes of cellular death, division, and immune recognition in stem cells.

A stem cell is an undifferentiated cell which can give rise to a succession of mature functional cells. A hematopoietic stem cell, for example, may give rise to any of the different types of terminally differentiated blood cells. Embryonic stem (ES) cells are derived from the embryo and are pluripotent, thus possessing the capability of developing into any organ or tissue type or, at least potentially, into a complete embryo.

One use for stem cells is in transplantation. When culture conditions are manipulated stem cells can be induced to differentiate to specific cell types, such as blood cells, neurons, or muscle cells. These differentiated cells may then be transplanted into a subject to treat specific diseases, such as hematopoietic disorders, endocrine deficiencies, degenerative neurological disorders.

Thus, in some aspects the invention involves a method for inducing expression of B7 on stem cells. This can be accomplished using activators of ROS and maintenance of growth conditions. In addition to the compounds that do not inhibit mitochondrial electron transport, the stem cells may be contacted with a compound that inhibits mitochondrial electron transport to induce co-stimulatory molecule expression. One compound for inhibiting mitochondrial electron transport is UCP. Mitochondrial UCP may be added or induced in the stem cell to induce co-stimulatory molecules. Preferably the mitochondrial UCP is an isolated molecule.

An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

Alternatively, when co-stimulatory molecule expression is reduced antigen specific immune responses may be suppressed, such as in the growth inhibited state describe above. Methods of achieving a growth inhibited state are useful, for instance, in the treatment of autoimmune disease and the prevention of graft and transplant rejection. In such a state co-stimulatory molecule expression is decreased or abolished and the cell is not recognized by the immune system.

Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis (MS), systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, auto immune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

For example, in a disease such as MS T cells attack the myelin sheath of neurons, resulting in destruction of the cell. By causing neuronal cells to assume a growth inhibitory state as described herein, the cells will avoid attack by T cells. This can be accomplished in vivo or in neural or stem cells being transplanted into a subject. These methods are also useful for promoting the successful transplantation of other organs into recipients without attack by the immune system. For example, donor organs can be treated with ROS inhibitors under conditions such as the use of fat for fuel to reduce expression of co-stimulatory molecule expression. When such tissues are implanted, they are not recognized by the immune system and will not be rejected.

Additionally, the methods of the invention may be used to generate an immune targeted state. Such methods are useful, for instance for treating cancer or infectious disease or preventing cancer or infectious disease (e.g., reducing a risk of developing cancer or infectious disease) in a subject at risk of developing a cancer or infectious disease. The cancer may be selected from the group consisting of biliary tract cancer, breast cancer, cervical cancer, choriocarcinoma, colon cancer, endometrial cancer, gastric cancer, intraepithelial neoplasms, lymphomas, liver cancer, lung cancer (e.g. small cell and non-small cell), melanoma, neuroblastomas, oral cancer, ovarian cancer, pancreatic cancer, prostate cancer, rectal cancer, sarcomas, thyroid cancer, and renal cancer, as well as other carcinomas and sarcomas. In some important embodiments, the cancer is selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer.

The methods of the invention are also useful for enhancing immune surveillance. When a co-stimulatory molecule is expressed on the surface of a cell in combination with an antigen presented in the context of MHC, antigen specific T cell proliferation and activation may be enhanced. Thus, by inducing levels of B7 expression on the surface of cells antigen specific immune responses may be enhanced. This is useful for treating disorders in which immune cell activation is desirable, such as infectious disease and cancer.

In some aspects of the invention the activator of ROS is administered to the subject or cell in conjunction with an antigen. This is useful, for instance, for treating a mammalian subject in vivo to induce an antigen-specific immune response. The term “in conjunction with” refers to delivery of the two components at the same time or different times, or in the same or separate vehicles. It is useful to produce antigen-specific immune responses against any foreign antigen whether it is capable of causing a pathological state or any damage to its mammalian host. The terms “foreign antigen” or “antigen” are used synonymously to refer to a molecule capable of provoking an immune response in a host, wherein the antigen is not a self-antigen. Thus, the term antigen or foreign antigen specifically excludes self-antigens. Self-antigens are used herein to refer to the peptide-antigens of autoimmune disorders. An immune response against the self-antigen results in an autoimmune disorder. The term self-antigen does not include, however, antigens such as cancer antigens, which are recognized by the host as foreign and which are not associated with autoimmune disease. Thus, the term antigen specifically excludes self-antigens and broadly includes any type of molecule (e.g. associated with a host or foreign cell) which is recognized by a host immune system as being foreign. Antigens include, but are not limited to, cancer antigens and microbial antigens and may be composed of cells, cell extracts, polysaccharides, polysaccharide conjugates, lipids, glycolipids, carbohydrates, peptides, proteins, viruses, viral extracts, etc.

A “cancer antigen”, as used herein, is a compound which is associated with a tumor or cancer cell surface and which is capable of provoking an immune response when expressed on the surface of an antigen-presenting cell in the context of a class II MHC molecule. Cancer antigens include but are not limited to Melan-A/MART-1, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-i, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, Ε-cadherin, α-catenin, β-catenin and γ-catenin, p120ctn, gp100^{Pmel117}, PRAME, NY-ESO-1, brain glycogen phosphorylase, SSX-1, SSX-2 (HOM-MEL-40), SSX-1, SSX-4, SSX-5, SCP-1 and CT-7, cdc27, adenomatous polyposis coli protein (APC), fodrin, PiA, Connexin 37, Ig-idiotype, pl5, gp75, GM2 and GD2 gangliosides, viral products such as human papilloma virus proteins, Smad family of tumor antigens, lmp-1, EBV-encoded nuclear antigen (EBNA)-1, or c-erbB-2.

In some embodiments, cancers or tumors escaping immune recognition and tumor-antigens associated with such tumors (but not exclusively), include acute lymphoblastic leukemia (etv6; aml1; cyclophilin b), B cell lymphoma (Ig-idiotype), glioma (Ε-cadherin; α-catenin; β-catenin; γ-catenin; p120ctn), bladder cancer (p21ras), billiary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-lA/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), hodgkins lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; cerbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100^{Pmel117}). These antigens are also useful according to the invention.

For examples of tumor antigens which bind to either or both MHC class I and MHC class II molecules, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol. 162:6849-6854, 1999; Boël et al., Immunity 2:167-175, 1995; Van den Eynde et al., J. Exp. Med. 182:689-698, 1995; De Backer et al., Cancer Res. 59:3157-3165, 1999; Jäger et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wölfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et at, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140, 1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J. Natl Cancer Inst 89:293-300, 1997; Coulie et al., Proc. Natl. Acad. Sci. USA 92:7976-7980, 1995; Wölfel et al., Science 269:1281-1284, 1995; Robbins et al., J. Exp. Med. 183:1185-1192, 1996; Brändle et al., J. Exp. Med. 183:2501-2508, 1996; ten Bosch et al., Blood 88:3522-3527, 1996; Mandruzzato et al., J. Exp. Med. 186:785-793, 1997; Guéguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999;Fisk et al., J. Exp. Med. 181:2109-2117, 1995; Brossart et al., Cancer Res. 58:732-736, 1998; Röpke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679, 1999. These antigens as well as others are disclosed in PCT Application PCT/US98/18601.

In other aspects, the antigen is a microbial antigen and the methods of the invention are useful for treating or preventing infectious disease. An infectious disease, as used herein, is a disease arising from the presence of a foreign microorganism in the body. A microbial antigen, as used herein, is an antigen of a microorganism and, includes but it not limited to, infectious virus, infectious bacteria, and infectious fungi.

Examples of infectious virus include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium such as Plasmodium falciparum, Plasmodium malariae, Plasmodium ovale, and Plasmodium vivax and Toxoplasma gondii.

The methods of the invention involve the use of activators of ROS and inhibitors of ROS. As used herein an “activator of ROS” is an agent which causes a local increase in ROS resulting in co-stimulatory molecule, such as B7 (and other related family members retaining sequence homology with B7) to be expressed on a cell surface. A subset of activators of ROS is neural cell ROS activators. A “neural cell ROS activator” as used herein is a compound or therapy that induces local reactive oxygen species in a nerve cell resulting in an induction of costimulatory molecule expression. These activators include but are not limited to reactive oxygen species, angiostatins, angiogenics, viral components, and exposure to sub-toxic microwaves or low dose radiation. Low dose radiation refers to radiation of less than 10 grey. In general an activator of ROS is a compound which inhibits mitochondrial electron transport and which causes dissipation of the mitochondrial proton motor force. These molecules include but are not limited to adriamycin, gamma interferon, bacterial byproducts such as lipopolysaccharides, lipoproteins BCG, fatty acids, cAMP inducing agents, a UCP expression vector, angiostatins, angiogenics, viral components (such as HIV Nef, HIV tat, and adenoviral El B), reactive oxygen species, and exposure to sub-toxic microwaves or low dose radiation. Although some of these compounds are known to be toxic in high doses, the therapies of the invention contemplate using such compounds in low doses which are not toxic. A “cAMP inducing agent” as used herein is any compound which elevates intracellular levels of cAMP. Such compounds include but are not limited to isoproterenol, epinephrine, norepinephrine, phosphodiester inhibitors, theophylline, and caffeine. For purposes of the patent application the term activator of ROS also refers to ROS (such as H₂O₂), which can be applied directly to cells. Other activators of ROS which are not inhibitors of mitochondrial electron transport include but are not limited to inhibitors of glutathione and glutathione S reductase, inhibitors of Copper/Zinc superoxide dismutase and Manganese superoxide dismutase, and inhibitors of lysosomal UCP. In yet other aspects of the invention, and in particular when the activator of ROS is administered with an antigen for the purposes of treating infectious disease, the activator of ROS does not include an inhibitor of lysosomal UCP.

Microwave radiation has been used in the past in toxic doses to heat cancer cells and cause localized death of heated cells. It has been discovered that microwave radiation can be applied to a variety of tissues including tumor and normal tissue in sub-toxic doses to induce intracellular reactive oxygen levels in those cells. These sub-toxic doses can be utilized to achieve the appropriate levels of reactive oxygen resulting in expression of co-stimulatory molecules under the conditions described above to produce cells in a growth induced state or an immune targeted state. Thus, microwave radiation can be used in combination with other factors to promote tissue generation or to promote immune recognition of cells. For instance breast cancer cells exposed to 10 GHz microwave radiation (2 mWatts) result in increases in metabolic and immune recognition levels (e.g. co-stimulatory molecule expression). Microwave radiation in a range of about 5 to about 50 GHz is sufficient to induce expression of co-stimulatory molecules required for T lymphocyte activation. A variety of ferromagnetic equipment can be used to generate the microwave radiation.

2-deoxyglucose and analogs thereof may be used to promote the effectiveness of low frequency/intensity microwaves in order to alter the expression of co-stimulatory molecules. 2-deoxyglucose competes with nutritional sugars (e.g. glucose) metabolically causing a dysfunction in metabolic mitochondrial events. Analogs of 2-deoxyglucose are compounds which are structurally similar and which also function to compete with nutritional sugars in metabolic processes.

An “amount of an activator of ROS effective to induce the expression of co-stimulatory molecules on the surface of the cell” as used herein, refers to an amount which is effective to cause an increase in ROS in the local area of the cell. Preferably the amount is that amount which is necessary to induce the expression of at least a single co-stimulatory molecule on the cell surface.

An “inhibitor of ROS” as used herein refers to an agent which causes a local decrease in ROS resulting in a decrease in co-stimulatory molecule expression on a cell surface. Inhibitors of ROS include but are not limited to compounds which activate or induce glutathione S reductase, glutathione, Copper/Zinc superoxide dismutase, or Manganese superoxide dismutase.

The cell is contacted with the activator or inhibitor of ROS to cause modulation of co-stimulatory molecules on the surface. As used herein, the step of contacting the cell with an activator or inhibitor of ROS can be performed by any means known in the art. For instance, if the activator or inhibitor of ROS is applied in vitro, it may simply be added as part of the cellular medium to a tissue culture dish of cells. If the method is performed in vivo, then the step of contacting may be performed by administering the activator or inhibitor of ROS by commonly used therapeutic techniques, such as parenteral administration, oral administration, or local administration. Other methods are well known to those of ordinary skill in the art.

Optionally a nucleic acid, such as a UCP or co-stimulatory molecule or receptor thereof can be delivered to a cell such that a peptide encoded for by the nucleic acid will be expressed in a cell in order to produce cells or reagents useful according to the invention. These methods may be accomplished using expression vectors which are prepared and inserted into cells using routine procedures known in the art. These procedures are described in more detail in co-pending patent application U.S. Ser. No. 09/277,575, having common inventorship, which is hereby incorporated by reference. Nucleic acids encoding UCP, co-stimulatory molecules and receptors thereof are known in the art and may be found in many references as well as in genbank under various accession numbers. The nucleic acid used will depend on the purpose of generating the expression vector useful in the methods of the invention. Those of skill in the art will be able to select the appropriate nucleic acid for expression. For instance, when it is desirable to express UCP in a mitochondria of a cell to promote uncoupling of the mitochondria, any of the UCP nucleic acids may be selected. UCP2 may be a preferred nucleic acid.

The nucleic acids useful herein may be operably linked to a gene expression sequence which directs the expression of the nucleic acid within a eukaryotic cell. The “gene expression sequence” is any regulatory nucleotide sequence, such as a promoter sequence or promoter-enhancer combination, which facilitates the efficient transcription and translation of the nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, and actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined nucleic acid. The gene expression sequences optionally include enhancer sequences or upstream activator sequences as desired.

The nucleic acid sequence and the gene expression sequence are said to be “operably linked” when they are covalently linked in such a way as to place the transcription and/or translation of the coding sequence under the influence or control of the gene expression sequence. If it is desired that the sequence be translated into a functional protein, two DNA sequences are said to be operably linked if induction of a promoter in the 5′ gene expression sequence results in the transcription of the sequence and if the nature of the linkage between the two DNA sequences does not (1) result in the introduction of a frame-shift mutation, (2) interfere with the ability of the promoter region to direct the transcription of the sequence, or (3) interfere with the ability of the corresponding RNA transcript to be translated into a protein. Thus, a gene expression sequence would be operably linked to a nucleic acid sequence if the gene expression sequence were capable of effecting transcription of that nucleic acid sequence such that the resulting transcript might be translated into the desired protein or polypeptide.

In other aspects of the invention the activator of ROS is a lysosomal UCP inhibitor. In preferred aspects of the invention the activator of ROS is not a lysosomal inhibitor when the activator is not administered to a cell for the purpose of treating an infectious disease, cancer or in conjunction with an antigen.

A “lysosomal UCP inhibitor” is any molecular species that prevents UCP activity in the lysosome. The lysosomal UCP inhibitor may function by preventing the activity of an expressed UCP, preventing the transcription of a lysosomal UCP gene, preventing the processing or translation of a lysosomal UCP RNA or preventing the processing, trafficking, or activity of a lysosomal UCP protein when administered in vivo or in vitro to a mammalian cell which is otherwise competent to express active lysosomal UCP. Thus, for example, lysosomal UCP inhibitors include lysosomal targeted nucleotides, nucleotide analogs, and binding peptides, repressors which prevent induction and/or transcription of the lysosomal UCP gene, antisense sequences which selectively bind to lysosomal UCP DNA or RNA sequences and which prevent the transcription or translation of the lysosomal UCP gene, competitive and non-competitive inhibitors of the activity of the lysosomal UCP protein. In some embodiments of the invention the lysosomal UCP inhibitor is a lysosomal UCP binding molecule or a lysosomal UCP antisense molecule. UCP binding proteins include for instance anti-UCP antibodies, including fragments of antibodies, such as FMC. These peptides are targeted to the lysosomal membranes in order to selectively bind to and inhibit the activity of lysosomal UCP. Other types of inhibitors include ribozymes which interfere with the transcription, processing, or translation of lysosomal UCP mRNA. In other embodiments the UCP inhibitor is a nucleotide or nucleotide analog targeted to the lysosome. These nucleotides and analogs are those described above, such as ATP.

Another preferred lysosomal UCP inhibitor is tunicamycin. Tunicamycin promotes intracellular trafficking of the lysosomal UCP from the intracellular location to the plasma membrane. When cells are administered tunicamycin the UCP is selectively targeted away from the lysosome, promoting respiratory burst and promoting antigen presentation.

In some aspects of the invention the lysosomal inhibitors are antisense oligonucleotides that selectively bind to a lysosomal UCP nucleic acid molecule or dominant negative UCP used to reduce the expression of lysosomal UCP. Antisense oligonucleotides are useful, for example, for inhibiting lysosomal UCP in a cell in which it is ordinarily expressed in the lysosome.

As used herein, the term “antisense oligonucleotide” or “antisense” describes an oligonucleotide which hybridizes under physiological conditions to DNA comprising a particular gene or to an RNA transcript of that gene and, thereby, inhibits the transcription of that gene and/or the translation of the mRNA. The antisense molecules are designed so as to hybridize with the target gene or target gene product and thereby, interfere with transcription or translation of the target mammalian cell gene. Those skilled in the art will recognize that the exact length of the antisense oligonucleotide and its degree of complementarity with its target will depend upon the specific target selected, including the sequence of the target and the particular bases which comprise that sequence. The antisense must be a unique fragment. A unique fragment is one that is a ‘signature’ for the larger nucleic acid. It, for example, is long enough to assure that its precise sequence is not found in molecules outside of the UCP gene. As will be recognized by those skilled in the art, the size of the unique fragment will depend upon its conservancy in the genetic code. Thus, some regions of genes, will require longer segments to be unique while others will require only short segments, typically between 12 and 32 base pairs (e.g. 12, 13, 14, 15, 16, 17, 18, 19, 20, 21, 22, 23, 24, 25, 26, 27, 28, 29, 30, 31 and 32 bases long).

It is preferred that the antisense oligonucleotide be constructed and arranged so as to bind selectively with the target under physiological conditions, i.e., to hybridize substantially more to the target sequence than to any other sequence in the target cell under physiological conditions. Based upon the known sequence of a gene that is targeted for inhibition by antisense hybridization, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 7 and, more preferably, at least 15 consecutive bases which are complementary to the target. Most preferably, the antisense oligonucleotides comprise a complementary sequence of 20-30 bases. Although oligonucleotides may be chosen which are antisense to any region of the gene or RNA (e.g., mRNA) transcripts, in preferred embodiments the antisense oligonucleotides are complementary to 5′ sites, such as translation initiation, transcription initiation or promoter sites, that are upstream of the gene that is targeted for inhibition by the antisense oligonucleotides. In addition, 3′-untranslated regions may be targeted. Furthermore, 5′ or 3′ enhancers may be targeted. Targeting to mRNA splice sites has also been used in the art but may be less preferred if alternative mRNA splicing occurs. In at least some embodiments, the antisense is targeted, preferably, to sites in which mRNA secondary structure is not expected (see, e.g., Sainio et al., Cell Mol. Neurobiol., (1994) 14(5):439-457) and at which proteins are not expected to bind. The selective binding of the antisense oligonucleotide to a mammalian target cell nucleic acid effectively decreases or eliminates the transcription or translation of the mammalian target cell nucleic acid molecule. Reduction in transcription or translation of the nucleic acid molecule is desirable in preparing an animal model for further defining the role played by the mammalian target cell nucleic acid in modulating an adverse medical condition.

The invention also includes the use of a “dominant negative lysosomal membrane UCP” polypeptide. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide as used herein in a cell is a reduction in lysosomal membrane expressed UCP. One of ordinary skill in the art can assess the potential for a dominant negative variant of a protein, and using standard mutagenesis techniques to create one or more dominant negative variant polypeptides. For example, one of ordinary skill in the art can modify the sequence of the lysosomal membrane UCP by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity, or simply for presence in the lysosomal membrane. Other similar methods for creating and testing dominant negative variants of a protein will be apparent to one of ordinary skill in the art.

The terms “treat” and “treating” as used herein refer to includes preventing the development of a disease, reducing the symptoms of disease, and/or inhibiting the progression of a disease, such as the growth of an established cancer.

The compositions useful in the invention may be formulated or unformulated. In general, the delivery formulations useful in the invention are divided into two classes: colloidal dispersion systems and biological vectors.

As used herein, a “colloidal dispersion system” refers to a natural or synthetic molecule, other than those derived from bacteriological or viral sources, capable of delivering to and releasing the composition in a subject. Colloidal dispersion systems include macromolecular complexes, nanocapsules, microspheres, beads, and lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 can encapsulate large macromolecules within the aqueous interior and these macromolecules can be delivered to cells in a biologically active form (Fraley, et al., Trends Biochem. Sci., 6:77 (1981)).

Lipid formulations for transfection are commercially available from QIAGEN, for example as EFFECTENE™ (a non-liposomal lipid with a special DNA condensing enhancer) and SUPER-FECT™ (a novel acting dendrimeric technology) as well as Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985), which is hereby incorporated by reference.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing an exogenous gene under the control of an appropriate promotor. The polymeric matrix is used to achieve sustained release of the exogenous gene in the patient. In accordance with the instant invention, the compositions of the invention described herein are encapsulated or dispersed within the biocompatible, preferably biodegradable polymeric matrix disclosed in PCT/US/03307.

The polymeric matrix preferably is in the form of a microparticle such as a microsphere (wherein the composition is dispersed throughout a solid polymeric matrix) or a microcapsule (wherein the composition is stored in the core of a polymeric shell). Other forms of the polymeric matrix for containing the composition include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and composition are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the chemical/physical vector is a biocompatible microsphere that is suitable for oral delivery. Such microspheres are disclosed in Chickering et al., Biotech. And Bioeng., (1996) 52:96-101 and Mathiowitz et al., Nature, (1997) 386:.410-414.

It is also envisioned that certain compounds useful in the invention may be delivered to the subject in a biological vector which is a nucleic acid molecule which encodes for a particular protein, such as UCP or a co-stimulatory molecule that is desirable to express in vivo. The nucleic acid encoding the protein is operatively linked to a gene expression sequence which directs the expression of the nucleic acid within a eukaryotic cell, as described above.

Compaction agents also can be used alone, or in combination with, a vector of the invention. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver the compositions in a form that is more efficiently taken up by the cell or, more preferably, in combination with one or more of the above-described vectors.

Other exemplary compositions that can be used to facilitate uptake by a target cell of the compositions of the invention include calcium phosphate and other chemical mediators of intracellular transport, microinjection compositions, electroporation and homologous recombination compositions (e.g., for integrating a composition of the invention into a preselected location within the target cell chromosome).

The pharmaceutical preparations of the invention are administered to subjects in effective amounts. An effective amount means that amount necessary to delay the onset of, inhibit the progression of, halt altogether the onset or progression of or diagnose the particular condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

Generally, doses of active compounds will be from about 0.01 mg/kg per day to 1000 mg/kg per day. It is expected that doses range of 50-500 mg/kg will be suitable, in one or several administrations per day. In the event that a response in a subject is insufficient at the initial doses applied, higher doses (or effectively higher doses by a different, more localized delivery route) may be employed to the extent that patient tolerance permits. Multiple doses per day are contemplated to achieve appropriate levels of compounds.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. As used herein, the compositions of the invention may include various salts.

The compositions of the invention may be combined, optionally, with a pharmaceutically-acceptable carrier. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the compositions of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the compositions of the invention into association with a carrier which constitutes one or more accessory ingredients. In general, the compositions are prepared by uniformly and intimately bringing the compositions of the invention into association with a liquid carrier, a finely divided solid carrier, or both, and then, if necessary, shaping the product.

Compositions suitable for oral administration may be presented as discrete units, such as capsules, tablets, lozenges, each containing a predetermined amount of the compositions of the invention. Other compositions include suspensions in aqueous liquids or non-aqueous liquids such as a syrup, elixir or an emulsion.

Other delivery systems, such as the vectors and delivery formulations described above may be used. One preferred delivery system can include time-release, delayed release or sustained release delivery systems. Such systems can avoid repeated administrations of the compositions of the invention described above, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the compositions of the invention is contained in a form within a matrix such as those described in U.S. Pat. Nos. 4,452,775, 4,667,014, 4,748,034 and 5,239,660 and (b) difusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

### EXAMPLES

### Example 1

### Use of Low Intensity Microwaves (200 mWat 10 GHz) to Induce Co-stimulatory Molecule Expression on Cells.

Methods: MCF7 cells were kept under standard tissue culture techniques utilizing complete RPMI medium and in a 37 degree CO₂ incubator. Prior to an experiment cells were counted. Depending on each experiment and the number of tests that were to be done post microwaves, between 25 and 50 million cells were harvested. These cells were placed into microfuge tubes according to the time of exposure: zero, 30 minutes, 1 hour, and 3 hours.

The cells were then exposed to 200 mW of low frequency microwaves for 30 minutes, 1 hour, or 3 hours. Cells were then stained with DCF-DA (Molecular Probes, Inc. Eugene, Oreg.) as an indicator of relative levels of H₂0₂. Cells were also stained with fluorescent labeled antibodies to human B7.2 (Pharmingen, Inc.), versus fluorescent labeled, isotype matched control antibodies, upper panels, left and right, respectively. Indicated levels represent the ratio of specific B7.2 stain over the fluorescently labeled isotype control for each condition. Replicate samples, microwaved for the indicated time periods, were subsequently cultured for an additional 21 hours and stained for H₂0₂ and B7.2, as indicated, lower panels. Cells were analyzed flow cytometrically using a Coulter Excel Flow Cytometer. Data were analyzed using Becton Dickinson CellQuest software. These data are representative of at least three repeated experiments.

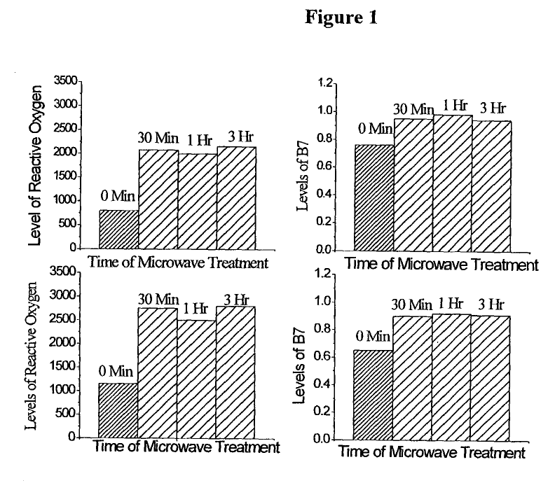

Results: Low frequency, low intensity microwaves induced increases in intracellular reactive oxygen and increases in cell surface expression of B7.2 on MCF7, human breast cancer cell lines. The intensity level of the microwaves was sufficiently low that the cells were viable and their temperature remained unchanged. As shown in FIG. 1, an increase in intracellular reactive oxygen (H₂O₂) in breast cancer cells (MCF7) exposed to the sub-toxic doses of microwave was observed. Additionally an increased level of the costimulatory molecule B7.2 was expressed at the cell surface. Similar experiments on leukemic cells (HL60 and U937) also showed substantial increase in B7.2.

### Example 2

### H₂0₂ Induces Co-stimulatory Molecule Expression on Cells.

Methods: Human myelocyte cells U937, HL60, PC12, and PC12Trk cells were cultured in the presence of subcytotoxic doses (0.25 mM) of H₂0₂ for 48 hours, harvested and stained with isotype controls or fluorescent labeled anti-B7.2 as described in Example 1. Cells were analyzed flow cytometrically using a Coulter Excel Flow Cytometer. Data were analyzed using Becton Dickinson CellQuest software. These data are representative of at least three repeated experiments.

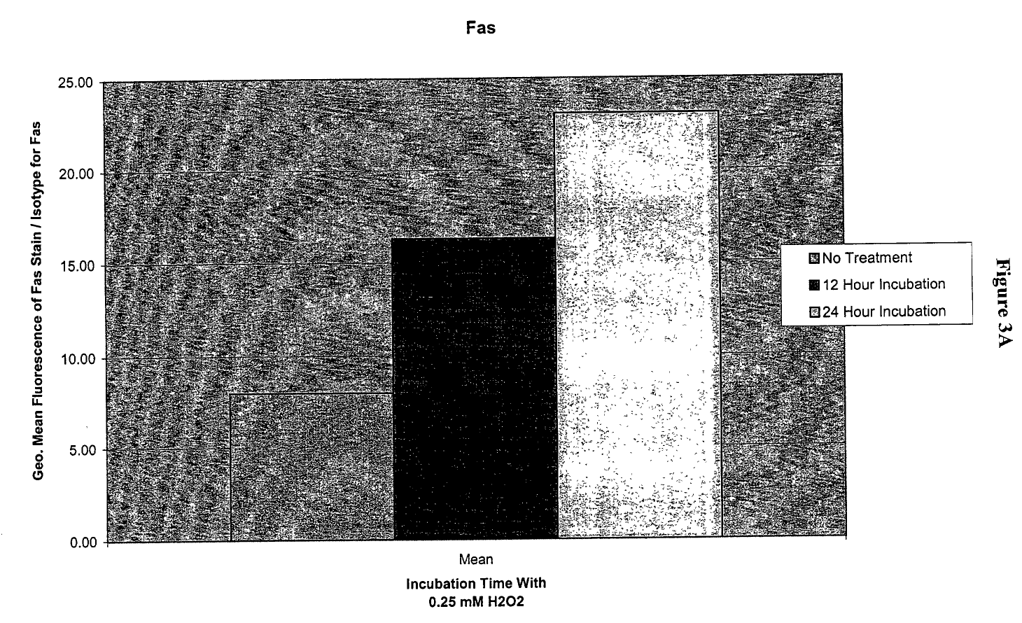

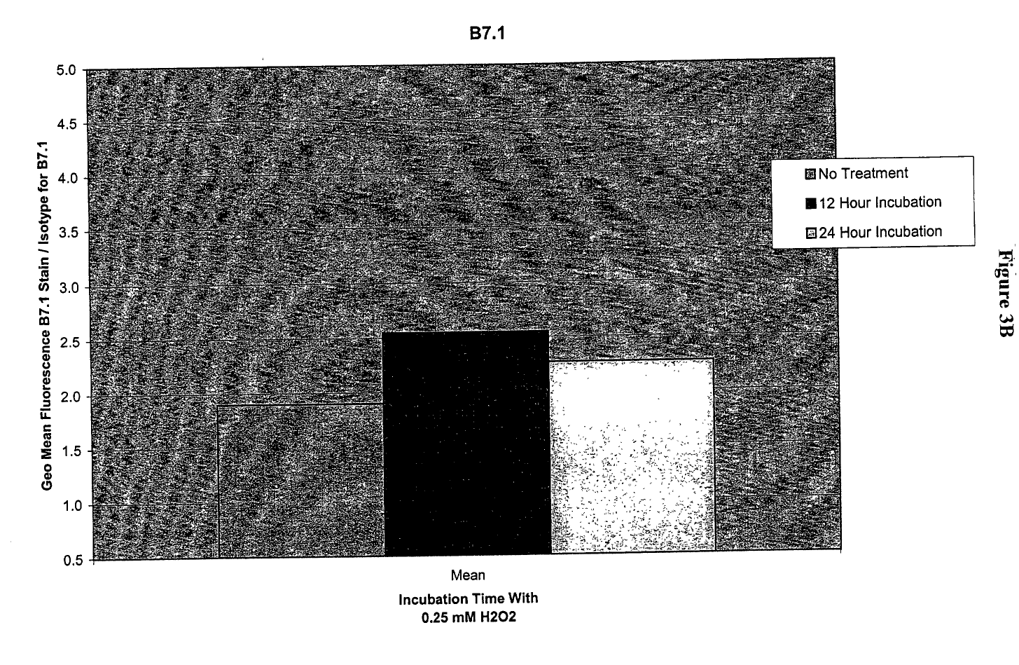

A mouse keratinocyte cell line was incubated with or without 0.25 mM Hydrogen Peroxide (H2O2) for 12 or 24 hours. Then the level of B7.1 and Fas were evaluated. The data is attached as FIGS. 3A and 3B.

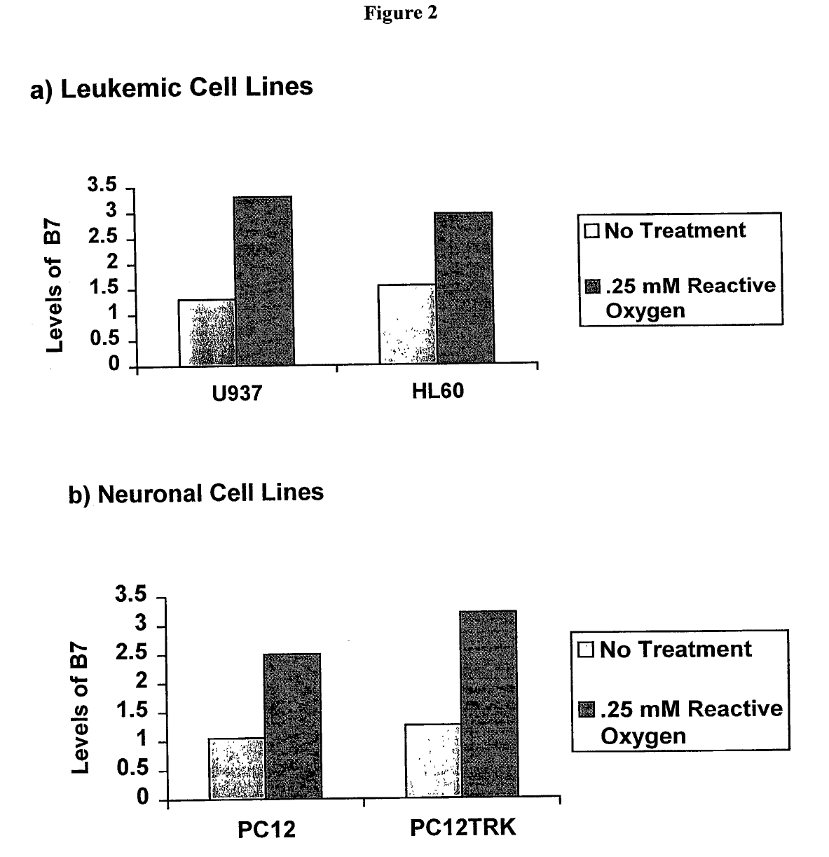

Results: Exogenous H₂0₂ directly induced increased cell surface expression of B7.2 on pro-myelocyte lines U937 and HL60 cells and in neural PC12 and PC12Trk neural cell lines. We added H₂O₂ (at subcytotoxic levels) directly to cells (MCF7, HL60, U937, and neural cells PC12 and PC12Trk¹⁵) in culture to see if this caused changes in B7 expression. The addition of H₂O₂ resulted in an increase in B7 in all cases, demonstrating that H₂O₂ does, in fact, causally produce immunologically important changes. In FIG. 2 we present representative data for the leukemic and neural cell lines showing substantial increases in level of B7 after H₂O₂ treatment. In FIGS. 3A and 3B it is demonstrated that H₂O₂ increases he level of expression of both B7.1 and Fas in keratinocytes.

### Example 3

### Insulin and Glucose Deprivation Reduce Levels of Co-stimulatory Molecule Expression.

Methods: HL60 cells were incubated with insulin or under in conditions of low glucose overnight. Cells were then harvested, stained with fluorescent labeled anti-Fas, or with isotype controls as indicated. Cells were analyzed flow cytometrically using a Coulter Excel Flow Cytometer. Data were analyzed using Becton Dickinson CellQuest software. These data are representative of at least three repeated experiments.

Results: Addition of insulin or removal of glucose result in loss of cell surface Fas expression. Cell metabolism must be responsive to a surplus or a deficit of nutrients. It has been discovered that the expression of Fas and B7 is responsive to insulin, high levels of glucose, and fatty acids. Insulin, for example, makes many changes in metabolic behavior. The addition of insulin to cells in culture can reduce levels of Fas ten-fold. Similarly, cells where glucose cannot bind to its receptor (for example in the presence of 2-deoxyglucose) show substantial reductions in these molecules. In contrast, when glucose levels rise beyond normal, both levels of intracellular reactive oxygen and Fas levels increase.

### Example 4

### Environmental Stress Such as Alcohol can Increase Reactive Oxygen Which Increases Levels of Co-stimulatory Molecule Expression.

Methods: Neural stem cells were treated as described above.

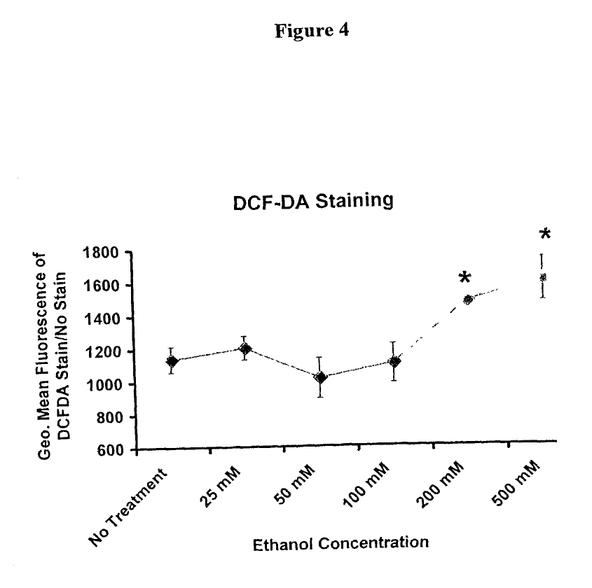

Results: As shown in FIG. 4 environmental stress such as alcohol can increase reactive oxygen, which results in increased induction of co-stimulatory molecules.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for promoting nerve cell generation, comprising:
contacting a nerve cell with a neural cell ROS activator in an effective amount to promote differentiation or growth.

2. The method of claim 1, wherein the neural cell ROS activator is selected from the group consisting of reactive oxygen species, angiostatins, angiogenics, viral components, and exposure to sub-toxic microwaves or low dose radiation.

3. The method of claim 1, further comprising contacting the nerve cell with a neural activating cell.

4. The method of claim 1, wherein the nerve cell is in vitro.

5. The method of claim 1, further comprising maintaining the nerve cell under growth conditions, wherein the conditions include exposure to at least one of nerve growth factor, fibroblast growth factor, and cytokines such as IL-2, IL-4, γ interferon, α, and β interferons, TNF (tumor necrosis factor) α, TGF (T-cell growth factor) α and β, and lymphotoxin.

6. The method of claim 1, further comprising contacting the nerve cell with a receptor for a co-stimulatory molecule.

7. A method for promoting non-neural tissue generation, comprising:
contacting a non-neural tissue with an activator of ROS in an effective amount to induce co-stimulatory molecule expression on the surface of cells of the tissue, and
exposing the tissue to growth conditions to promote generation of the tissue.

8. The method of claim 7 wherein the ROS activator is selected from the group consisting of γ interferon, lipoproteins, fatty acids, cAMP inducing agents, a UCP expression vector, a B7.1, B7.2 or CD40 expression vector, angiostatins, angiogenics, viral components, and exposure to sub-toxic microwaves or low dose radiation.

9. The method of claim 7, further comprising exposing the non-neural tissue to a T cell.

10. The method of claim 9, wherein the non-neural tissue is exposed to the T cell in vitro.

11. The method of claim 9, wherein the non-neural tissue is implanted in a subject after exposure to the T cell.

12. The method of claim 11, wherein the T cell is a cell of the subject.

13. The method of claim 12, wherein the non-neural tissue is autologous tissue.

14. The method of claim 12, wherein the non-neural tissue is a donor organ.

15. The method of claim 7, wherein a biopsy of the non-neural tissue is removed from a subject and wherein the biopsy of non-neural tissue is exposed to a T cell of the subject.

16. The method of claim 7, wherein the growth conditions include exposure to at least one of insulin, fibroblast growth factor, platelet derived growth factor, erythropoietin, and cytokines such as IL-2, IL-4, γ interferon, α and β, interferons, TNF (tumor necrosis factor) α, TGF (T-cell growth factor) α and β, and lymphotoxin.

17. A method for transplanting an organ into a recipient subject, comprising,
treating a donor organ with an inhibitor of ROS in an effective amount to reduce costimulatory molecule expression on cells of the donor organ, and transplanting the donor organ into the recipient subject.

18. The method of claim 17, wherein the inhibitor of ROS is selected from the group consisting of compounds which activate or induce glutathione S reductase, glutathione, Copper/Zinc superoxide dismutase, and Manganese superoxide dismutase.

19. A method for treating cancer, comprising:
exposing cancer cells of a subject to sub-toxic levels of microwave or to sub-toxic levels H₂O₂ in an effective amount to induce expression of a co-stimulatory molecule on the surface of the cancer cells and contacting the cell with an agent to kill the cell in order to treat the cancer.

20. The method of claim 19, further comprising exposing the cancer cells to 2-deoxyglucose or analogs thereof.

21. The method of claim 19, wherein the agent is a co-stimulatory molecule receptor.

22. The method of claim 21, wherein the co-stimulatory molecule receptor is on an immune cell.

23. The method of claim 21, wherein the co-stimulatory molecule receptor is a soluble receptor.

24. A method for inhibiting co-stimulatory molecule expression in a cell for in vivo transplantation, comprising:
contacting a cell with an inhibitor of ROS to inhibit co-stimulatory molecule expression in the cell, and implanting the cell in a subject.

25. The method of claim 24, wherein the cell is a stem cell.

26. The method of claim 24, wherein the co-stimulatory molecule is B7.1, B7.2 or CD40.

27. The method of claim 24, wherein the cell is selected from the group of cells consisting of kidney, lung, pancreas, skin, liver, eye, ovary, testes, and Sertoli cells.

28. The method of claim 24, further comprising administering the stem cell to a subject.

29. The method of claim 24, wherein the cell is grown in vitro under growth conditions prior to implantation.

30. The method of claim 28 wherein the inhibitor of reactive oxygen species is a compound selected from the group consisting of glutathione S reductase, glutathione, Copper/Zinc superoxide dismutase, and Manganese superoxide dismutase

31. A method for inducing co-stimulatory molecule expression in a growth induced cell, comprising:
contacting a cell with an activator of ROS to induce co-stimulatory molecule expression in the cell, and exposing the cell to growth conditions to promote cell proliferation.

32. The method of claim 31, wherein the growth conditions include exposure to at least one of insulin, nerve growth factor, fibroblast growth factor, platelet derived growth factor, erythropoietin, and cytokines such as IL-2, IL-4, γ interferon, α and β interferons, TNF (tumor necrosis factor) α, TGF (T-cell growth factor) α and β, and lymphotoxin.

33. The method of claim 31, wherein the co-stimulatory molecule is B7.1, B7.2 or CD40.

34. The method of claim 31, wherein the method is performed in vitro.

35. The method of claim 31, wherein the activator of ROS a reactive oxygen species.

36. The method of claim 34, further comprising administering the cell to a subject.

37. The method of claim 31, further comprising contacting the cell with an antigen.

38. The method of claim 31, wherein the method is performed in vivo in a subject.

39. The method of claim 31, wherein the activator of ROS is an inhibitor of mitochondrial electron transport selected from the group consisting of reactive oxygen species, angiostatins, angiogenics, viral components, and exposure to sub-toxic microwaves or low dose radiation.

40. The method of claim 39, wherein the viral component is a gene product selected from the group consisting of HIV Nef, HIV tat, and adenoviral E1B.

41. The method of claim 31 wherein the activator of ROS is an inhibitor of glutathione or glutathione S reductase.

42. The method of claim 31, wherein the activator of ROS is an inhibitor of superoxide dismutase.

43. The method of claim 31, wherein the activator of ROS is an inhibitor of lysosomal UCP.

44. The method of claim 31, wherein the activator of ROS is exposure to microwaves.

45. The method of claim 43, wherein the cell is a nerve cell.

46. The method of claim 31, wherein the cell is a neutrophil.

47. A method for modulating B7.1, B7.2 or CD40 expression on embryonic stem cells, comprising:
contacting an embryonic stem cell with a compound for modulating reactive oxygen species to modulate B7.1, B7.2 or CD40 expression on the embryonic stem cell.

48. The method of claim 47, wherein the compound for modulating reactive oxygen species is an inhibitor of ROS.

49. The method of claim 47, wherein the compound for modulating reactive oxygen species is a reactive oxygen species or an activator of ROS.

50. The method of claim 47, further comprising administering the embryonic stem cell to a subject.

51. A method for treating autoimmune disease, comprising,
administering to a subject having or at risk of developing an autoimmune disease an inhibitor of ROS in an effective amount to reduce costimulatory molecule expression on target autoimmune cells in order to treat the autoimmune disease.

52. The method of claim 51, wherein the inhibitor of ROS is selected from the group consisting of compounds which activate or induce glutathione S reductase, glutathione, Copper/Zinc superoxide dismutase, or Manganese superoxide dismutase.

53. The method of claim 51, wherein the autoimmune disease is multiple sclerosis.
